(19) United States
(12) Plant Patent Application Publication
(10) Pub. No.: US 2003/0115643 P1
(43) Pub. Date: Jun. 19, 2003

(54) Miniature rose plant named 'Savabusy'

(76) Inventor: John M. Saville, Newburyport, MA (US)

Correspondence Address: CHRISTIE, PARKER & HALE, LLP, 350 WEST COLORADO BOULEVARD, SUITE 500, PASADENA, CA, 91105, US

(21) Appl. No.: 10/021,857

(22) Filed: Dec. 17, 2001

(51) Int. Cl. A01H 5/00

(52) U.S. Cl. PLT/116

(57) ABSTRACT

A new and distinct Miniature Rose plant having attractive predominately orange flowers.

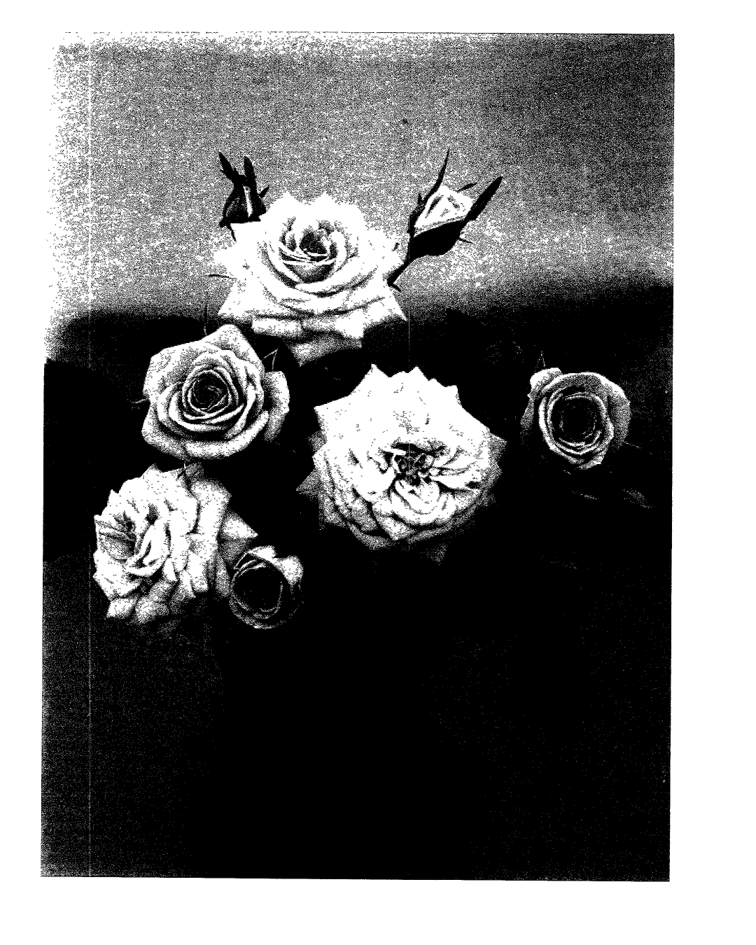

### BACKGROUND OF THE INVENTION

The present invention relates to a new and distinct variety of rose plant of the Miniature class. The new variety was created by crossing as seed parent the variety known as ‘MORSEGOLD’ (U.S. Plant Pat. No. 6,617) and as pollen parent, the variety known as ‘SAVASPARK’ (U.S. Plant Pat. No. 9,799). The varietal denomination of the new variety is ‘SAVABUSY’.

### SUMMARY OF THE INVENTION

Among the novel characteristics possessed by the new variety which distinguishes it from its parents and all other varieties of which I am aware, is its flower shape and flower color.

Asexual reproduction by propagation of vegetative cuttings of the new variety as performed in Essex County, Massachusetts show that the foregoing and other distinguishing characteristics come true to form and are established and transmitted through succeeding propagation.

### COMPARISON WITH PARENTS

The new variety may be distinguished from its parents in its form and flower color.

The new variety has predominately orange flowers whereas the flowers of its seed parent, are yellow and the flowers of the pollen parent are medium red.

### COMPARISON WITH RELATED VARIETIES

In addition to the foregoing, the new variety also differs from other cultivars of the same cross.

The first is ‘SAVABINO’ (U.S. Plant Pat. No. 10,980), ‘BAMBINO’) which is overall a considerably smaller plant with smaller blooms of a deeper orange red coloring.

The second is ‘SAVAPPLE’ (U.S. Plant patent application Ser. No. 09/727,065, filed Nov. 30, 2000) which is a lower growing plant and although the open blooms are about the same size, ‘SAVAPPLE’ has about half as many petals as this new cultivar and also is more red in its coloring.

### BRIEF DESCRIPTION OF ILLUSTRATION

The accompanying photograph illustrates a typical specimen of the new variety and shows the flowering thereof from bud to full bloom depicted in color as nearly true as it is reasonably possible to make in a color illustration of this character. Throughout this specification, color references and/or values are based upon The Royal Horticultural Society Colour Chart and terminology used in the color descriptions herein refers to plate numbers in this color chart except where common terms of color definition are employed.

### DESCRIPTION OF THE NEW VARIETY

The following observations were made of 6-year old plants of the new variety grown outdoors, in 8 inch standard pots, in Essex County, Massachusetts during the month of September. Phenotypic expression may vary with environmental, cultural and climatic conditions, as well as differences in conditions of light and soil.

Flower:

Bud.—Medium size; length about 11/16 inch; width about 7/16 inch just before sepals divide Form: Obovate Color: When sepals first divide color near 170A and 170B towards the tip blending into near 14C; when petals begin to unfurl, color near 33B and near 14C reverse and between near 33B and near 35A toward the tip.

Sepals.—Lanceolate, extending ½ inch beyond the tip of the bud; the 3 outer most sepals having one “flag” on 1 or both margins Upper Surface: Texture glabrous; color near 144A, may be flushed in sun with 187C Under surface: Texture very fine down coating; color near 147C and near 138A at the tip.

Permanent.—Yes.

Hip/Stem position.—Rolling back to stem and remaining thus and attached to the receptacle Peduncle: Peduncle with receptacle and sepals die and remain attached to plant Length: About ¾ to 1 inch Aspect: Straight Strength: Erect Color: Near 144A, side facing sun may be flushed with 187C Texture: Extremely fine hairs.

Bloom: Size: Average when fully expanded just under 1½ inches Borne: Singly Form: Opens flat; petals becoming and remaining loosely rolled outward

Petalage.—Between 35 to 40.

Fragrance.—Slight, spicy.

Persistence.—Only the petals drop off cleanly between 7 to 9 days.

Lasting quality.—As cut flower, between 4 to 7 days.

Petals:

Texture.—Glabrous.

Appearance.—Inside velvety; outside satiny.

Form.—Wide spatulate.

Size.—Length about ⅝ inch at point of attachment to tip; outer petal width up to about ¾ inch at widest part.

Arrangement.—Imbricated — shingle-like; not regularly overlapped.

Color.—During first 2 days Inner Petals: Upper side between 33B and 35A; reverse between 33B and 35A at the tip then becoming near 9C Outer Petals: Upper side near 33C and blending into near 9C ⅔ of the way down the petal; reverse near 39A along the margin blending into near 9C When half-blown: Little change but more of the petals becoming the coloring of the outer petals.

Base of petals (Point of attachment).—Near 9C.

General tonality from a distance.—Medium orange; sometimes appears “glowing”.

Color change.—Lightens and becomes more pink; starting with outer petals first and then all becoming near 51C and the yellow gradually disappears.

General tonality from a distance.—Apricot-orange; color stays the same after 3 or more days.

Petaloids in center.—1 or more Size: Average width at widest part about ⅛ inch; length from point of attachment about ¼ inch Color: Same as inner petals Texture: Glabrous.

Notable characteristics.—None.

Reproductive Organs:

Arrangement of stamens, filaments and anthers.—Arranged regularly around outer edge of receptacle.

Stamens.—About 20, but often less.

Filaments.—Length about 1/16 inch; general color near 1C.

Anthers.—Barely discernable, color near 9C.

Pollen.—Very little; color near 17A.

Pistils.—About 30 or less.

Styles.—Uneven length, thin; between 2/16 and 3/16 inch; color near 145C and appearing translucent.

Stigmas.—Near 145C.

Receptacle.—Shape: Circular; surface texture glabrous Size: Diameter about 5/16 inch Color: Near 145C.

Hips.—None observed.

Plant:

Form.—Upright, compact.

Growth.—Very vigorous, well branched.

Size.—Height attained about 14 inches; width at maturity about 2 feet.

Length of flowering stem.—Most often about 3 inches; may be as short as 2 inches when borne singly.

Foliage.—Pinnately compound of 5 leaflets; occasionally 3 leaflets; rarely 7 leaflets Size: Mature leaflet is medium sized, about 3⅜ inches from stem to tip measured along the rachis; mature terminal leaflet is about 1¼ inches from base to tip Shape: Oval pointed Quantity: Abundant Color New foliage: Upper side near 137A; the very edge may be near 187C when in the sun; under side near 146A, may be flushed very lightly with near 187C Mature foliage: Upper side between 137A and 147A; under side near 147B Texture Upper side: Smooth, matte Under side: Smooth with slightly raised veins Edge: Serrated Serration: Fine and uneven.

Rachis.—Texture smooth Color Mature Leaf: Upper side color near 144B with edges the same as the foliage — between 137A and 147A; under side near 147B Immature Leaf: Upper side near 187A; under side near 146B, flushed with 187C Prickles: Hairs and occasionally prickles on under side of rachis of older leaves

Stipules.—Slightly bearded; length about 11/32 inch; color on mature leaf near 147D and near 139A on the tips.

Petioles.—Texture smooth; upper side color same as rachis, near 144B, under side near 144C.

Resistance to disease.—Resistance to Blackspot good; average resistance to Mildew; no resistance to Rust observed; resistance against all insects good.

Wood:

New shoots.—Bark: Smooth Wood: Near 144A, may be flushed with near 187C.

Old wood.—Bark: Smooth Wood: Near 146A.

Thorns.—Quantity: Few observed on main stalks from base; just more than average on laterals from stalk Form: Thin, straight, angled down to fine point; irregularly positioned Length: About ¼ inch Color Immature thorns: Near 147D and when exposed to sun between 178A and near 183B and only the tips being near 147D Mature thorns: Near 165B Prickles: No prickles observed on main or lateral stalks; some prickles observed on older leaves; color near 24D.

Winter hardiness.—Tested hardy to zones 4 to 9; winter protection recommended in Zones 5 and colder.

I claim:

1. A new and distinct Miniature rose plant of the variety substantially as shown and described.
